(19) United States
(12) Patent Application Publication
(10) Pub. No.: US 2017/0056579 A1
(43) Pub. Date: Mar. 2, 2017

(54) SPINDLE DEVICE FOR A PISTON OF A RESERVOIR WITH MEDICAMENT FLUID

(71) Applicant: MeaMedical AG, Riken (CH)

(72) Inventor: Alex MÜRI, Bremgarten b. Bern (CH)

(73) Assignee: MeaMedical AG, Riken (CH)

(21) Appl. No.: 15/350,197

(22) Filed: Nov. 14, 2016

Related U.S. Application Data
(63) Continuation of application No. PCT/EP2014/059889, filed on May 14, 2014.

(51) Int. Cl. A61M 5/145; F16H 25/24; F16H 25/20

(52) U.S. Cl. CPC A61M 5/1452; F16H 25/2056; F16H 25/2454

(57) ABSTRACT

A spindle device for a piston, held in a reservoir containing a medicament fluid. The spindle device includes: a first displacement stage with a thread, a second displacement stage with a thread, a drive stage between the displacement stages and having two threads, with one thread engaging the thread of the first displacement stage forming a first spindle drive, and the second thread engaging the thread of the second displacement stage forming a second spindle drive. The first displacement stage and the drive stage move simultaneously in the direction of advance. The first and second displacement stages are connected in a rotationally fixed manner by an element axially surrounding the drive stage for the rotational securing, and the drive stage can be coupled in a rotationally fixed manner to an entrainment rod of a drive device, with the entrainment rod insertable into an axial hole on the drive stage.

### TECHNICAL FIELD

The invention relates to a spindle device for a piston.

### BACKGROUND

Portable injection and/or infusion devices are frequently used to administer fluid medication, particularly insulin. In such cases, the medication fluid is transported continuously or practically continuously by means of a metering apparatus including a drive device for a piston and a reservoir that contains the fluid. The reservoir piston is moved and displaces medication fluid in the reservoir to be delivered to the patient. Such devices are used as pump systems and manually operated pens in insulin treatment. An injection pen is known from WO93/16740 for example. Whether they are used in injection pens or insulin pumps, however, they devices must be as compact and reliable as possible and must be completely safe for the user.

One example of an insulin pump is the D-TRONplus pump manufactured by Roche Diabetes Care GmbH. This has a spindle device consisting of three telescopic spindle stages and arranged in fixed manner inside the pump. But in this case, a first displacement stage, which is displaceable towards the reservoir piston, is only capable of linear motion. A second displacement stage is able to perform both a linear advancing motion and rotation, being entrained by a drive stage. The drive stage only performs a rotating motion, to generate the linear motion of the first or second displacement stage. The drive device with its permanently attached spindle device of the D-TRONplus pump is described in DE 197 17 107 B4. Besides the serial embodiment, in which the first and second displacement stages are arranged one behind the other and the drive stage drives the second displacement stage as a third spindle element, a spindle arrangement is also described in which the drive stage is arranged between a first displacement stage and a reaction stage. In this arrangement, the first displacement stage moves away from the drive stage, while the drive stage also moves away from the fixed reaction stage which is connected to a housing. This arrangement is intended for spindle devices that are mounted permanently inside the pump. It may be considered a parallel embodiment, since both movable stages—the first displacement stage and the drive stage—move in the advance direction at the same time, whereas in the serial embodiment mentioned earlier only a spindle stage is moved. Serial embodiments in which the extending drive stages are extended consecutively, as is the case with the D-TRONplus pump, have the disadvantage that the pumping action may be interrupted during the transition from the first extending stage to the second extending stage as a consequence of uncompensated play in the thread. This can result specifically in a reduced flow rate, which in turn means that the patient does not receive sufficient medication fluid. This problem is known, and a resistive element for this with the task of compensating for the thread play in the spindle drives in the direction of advance is disclosed in DE 100 15 175 A1. The drawback of this further resistive element, which is essential for compensating for thread play, consists in that it adds to the axial length of the spindle device and thus also of the insulin pump, and it increases the complexity of the drive device. In FIG. 1 of DE 100 15 175 A1 it is evident that further elements, such as sealing points located on the spindle device, a sealing point for the drive stage, limit stops for the spindle stages and a cover for the drive stage increase the structural size of the spindle device in the axial direction considerably. The drive device according to DE 100 15 175 A1 includes in corresponding manner a complex structure consisting of many components, which has a detrimental effect on the service life and reliability of the insulin pump. Furthermore, such a structure is expensive to manufacture and install.

For the embodiment of FIGS. 13 and 14 disclosed in DE 197 17 107 A1, the drive stage positioned between the first displacement stage and the reaction stage is driven by an external drive mechanism. The drive mechanism has an unfavourably large diameter, with the result that the friction losses are considerably in any mounting, which is formed chiefly by sealing points with o-rings. In order to prevent a motor for the embodiment of FIGS. 13 and 14 from coming into contact with water or some other fluid substance, the drive mechanism must be sealed both above and below a gearwheel connection created on the drive mechanism. Such a seal on a large diameter increases the friction losses substantially and affects the energy consumption of the metering apparatus unfavourably. The embodiment according to FIGS. 13 and 14 is therefore difficult to seal. The disadvantages described are taken into account for the purpose of producing the most compact pump possible according to DE 197 17 107 A1, which pump most importantly must have the smallest dimension possible in the longitudinal axis of the spindle device. The minimisation of the lengthwise dimension along the spindle axis corresponds to the problem to be solved as originally described in DE 197 17 107 A1. In the claims of the granted patent EP 0 991 440 B1, for which DE 197 17 107 A1 constitutes the priority application, it is evident that minimising the longitudinal axis represents the essential object of the invention. This is achieved with a design in which an external drive stage in the form of a drive sleeve accommodates the displacement stages. For this reason, in the embodiment of FIGS. 13 and 14 of DE 197 17 107 A1 the reaction stage of the spindle device is connected in fixed manner to a housing and is braced against a lower inner housing wall. To ensure that the dimension of the medication pump along the spindle axis is as small as possible, the drive mechanism of the drive stage is driven from the side, by the laterally mounted motor.

The essential axial overlap between the drive mechanism and the drive stage with a dog and the axial limit stops for the spindle stages whose purpose is to prevent the spindle stages from separating during operation are also disadvantageous. Both the overlap referred to above and the limit stops on the spindle stages increase the structural length of the pump along the spindle axis. Moreover, the embodiment of FIGS. 13 and 14 includes a rotational lock for the first displacement stage. This is constructed as a sleeve and is connected in non-rotatable manner to a base part. Viewed in the radial direction, the arrangement of FIG. 14 includes the three telescopic spindle stages, the drive mechanism, a cylindrical mounting conformed on the base part for the drive mechanism, and the rotational lock for the first displacement stage. Consequently, the radial dimension of this device is so large and cumbersome that this embodiment has not been put to use in practice as a compact insulin pump. It is also known that the drive device of the D-TRONplus, which is disclosed in FIG. 24 of DE 197 17 107 B4, is not protected from contaminants. Particularly the spindle drives and threads of the first and second displacement stages are not protected against contamination by dust, insulin, cleaning substances and water. Contaminants of such kind can shorten the service life of this spindle device arranged permanently on the housing. The embodiment according to FIGS. 13 and 14 exhibits significant friction losses and is therefore unsuitable from the point of view of energy, and it is difficult to insulate the motor gearing arrangement from outside influences. The drive device of the D-TRONplus pump of FIG. 24 includes a serial embodiment of the spindle device. On the other hand, the construction according to FIGS. 13 and 14 corresponds to the parallel embodiment described in the introduction.

Telescopic spindle devices of the serial design are known from WO 94/15660 and WO 97/00091. They are always constructed in the same way in their arrangement and have a first and a second displacement stage, wherein the first displacement stage surrounds the second displacement stage in the manner of sleeve and the second displacement stage surrounds a drive stage in the manner of sleeve. In this arrangement, the drive stage drives one of the displacement stages at a time. As soon as the extending displacement stage reaches its axial limit stop, the other displacement stage begins its movement. This arrangement has the unfavourable transition between the drive stages described earlier, in which it is possible that a lower flow rate of the medication fluid may occur. In WO 97/00091, a two-part rotational lock for the first displacement stage is represented, consisting of one fixed sleeve and one movable sleeve. This arrangement has three stages for the spindle device and two sleeves for the rotational lock, and has an unsuitable radial dimension, so that can only be used for larger ampoule volumes. The document WO 94/15660 includes a note to this effect, according to which the spindle device is provided for standard ampoules of “5 cc, 10 cc, etc.”. In the device disclosed in WO 97/00091, the ampoule body is attached to an intermediate part, and the lower element of the rotational lock is connected to the intermediate part in non-rotating manner via a magnet located on the intermediate part. In a further handling step, the intermediate part coupled to the ampoule must be connected to a drive device. In this step, not only must the intermediate part be attached to the drive device, but the driving gearwheel of the drive device must be brought into engagement with a driven gearwheel of the drive stage for the spindle device. It is quite evident that the construction of these embodiments is complicated, they comprise many components and require several handling steps. Furthermore, not means are provided for protecting the spindle device and the drive device from contaminants. In this context it was also found subsequently that the telescopic spindle devices according to the embodiments of WO 94/15660 and WO 97/00091 can be reused by a user. Since the driven gearwheel protrudes into the outside environment, so that a user may have direct access to the drive stage, the user can restore the spindle device to its starting position by rotating the drive stage backwards. For wearing parts which should be protected from contamination with bacteria and the like, and which should only be used once, such a solution is not safer for the user.

Other devices for delivering medication are also known from DE 28 09 990 C2, DE 34 32 152 C2 and DE 37 33 452 C2. A large number of single-stage spindle devices is also known. In devices of this kind, only one stage moves. Single-stage spindle devices are not optimal for very compact apparatuses because their dimension along the longitudinal axis of the spindle device is too large. Single-stage spindle devices are known from WO2009/125398 and EP0143895 are mentioned as examples of single-stage spindle devices here solely for the sake of completeness. It may be deduced from the prior art that in terms of compact devices for administering insulin an arrangement of the spindle device has established itself in which the axial length of the pump is minimised. In this arrangement, the drive stage is driven from the side via a gearwheel connection. For a person skilled in the art, this arrangement is obvious and preferred, because it enables him to displace the spindle starting from a rear wall of the housing, and in this way minimise the length thereof in the axial direction, which consists of the spindle device and the reservoir for the device. Accordingly, this construction is used both in DE 197 17 107 A1 for telescopic spindle devices and for example in WO2009/125398 and EP0143895 for single-stage spindle apparatuses. On the other hand, the instruments of WO 94/15660 and WO 97/00091 were not designed for compact devices for delivering insulin, so the driving motor thereof may be arranged axially below the spindle device.

### SUMMARY

One aspect of the invention relates to a spindle device for a piston of a reservoir that is replaceable and may be coupled to a drive device simply and which ensures safe and reliable operation for the patient. The spindle device should also be easily producible and have a small number of components. Moreover, it should not be possible for the user to return a used spindle device to a starting state himself and use it again.

Such a spindle device has two rotationally secured displacement stages and a drive stage arranged between the displacement stages, wherein a first spindle drive is constructed between the first displacement stage and the drive stage, and a second spindle drive rotating in the opposite direction is arranged between the drive stage and the second displacement stage. In a starting state, the displacement stages at least partially overlap each other. Since an element for the rotational lock that surrounds the drive stage axially connects the two displacement stages in non-rotating manner, and the drive stage of the spindle device can only be coupled in non-rotating manner directly or indirectly to an entrainment rod designed in the form of a coupling element by an opposing frontal face of the spindle device, the user is not able to return a used spindle device to a starting state thereof himself, wherein the entrainment rod may be introduced into an axial hole conformed on the drive stage. On the other hand, the user can easily replace the used spindle device with a new one.

These properties of the spindle device according to the invention prolong the service life of the entire metering apparatus, since the spindle device which is subject to wear can be replaced by the user. This in turn has the effect of prolonging the service life of the pump and improving safety for the user as a result of greater reliability and longer service life of the entire metering apparatus. The spindle device according to the invention has only one interface with the drive device. This interface between the drive device and the spindle device is provided by the coupling member arranged on the drive device. The coupling member of the drive device may be sealed easily on a housing of the drive device, which in turn may further increase the reliability and service life of the entire metering apparatus. In the coupled state, the drive stage arranged between the displacement stages is connected to the coupling member and enclosed by the surrounding element for the rotational lock. In the uncoupled state, the user has no access to the drive stage, because the drive stage is surrounded radially by the element of the rotational lock and it is only accessible and operable from below with the aid of appropriate tools. The spindle device according to the invention guarantees that, in order to avoid contamination the user must use a new spindle device for each new reservoir filled with medication fluid.

For the purposes of the present invention, the term indirect, non-rotatable coupling between the drive stage and the entrainment rod is understood to mean that an intermediate element may be provided between the drive stage and the entrainment rod or for example a two-part design of the entrainment rod may be provided by an additional guide for example.

Advantageous variations of the invention are also disclosed.

The drive stage is preferably formed by two cylindrical sleeves, wherein the sleeves are connected to each other in non-rotating manner by the upper frontal faces thereof, that is to say on the side closest to the piston, or by the lower frontal faces thereof, that is to say on the side farthest from the piston. The non-rotating connection is preferably created on the upper frontal faces, on the side closest to the piston.

The coupling member may preferably be designed as a profiled entrainment rod. In this context, the profile may be selected such that the user is not able to create a coupling with the drive stage using a screwdriver or some other tool. It is also advantageous if the entrainment rod protrudes as far as possible into the drive stage. In the starting state, the overlap should have at least a displacement path V of a displacement stage. This ensures that the entrainment rod and the drive stage remain engaged for the entire displacement path of the piston. The overlap of at least one displacement path V between the drive stage and the entrainment rod means that partly filled reservoirs can also be used. This is advantageous because not all users need the same quantity of medication fluid every day and some individuals may prefer to only partly fill the reservoir.

In a preferred embodiment the surrounding element for the rotational lock may have the form of a sleeve. Since both drive stage are formed by cylindrical elements, it is expedient to construct the surrounding element for a non-rotating connection between the two displacement stages from a cylindrical element as well. In this way, a spindle device may be created that consists entirely of cylindrical elements and so has a circular cross section. This arrangement is particularly advantageous for the dispensing from cylindrical reservoirs, because the radial dimension of such as spindle should be small. Such a spindle device is particularly well suited for dispensing prefilled insulin ampoules.

The spindle device may preferably be arranged directly on the piston. Then, the piston itself may be designed as the first displacement stage of the spindle device. If the piston and therewith also a wall of the reservoir have oval cross sections, the wall may be used as an element for the rotational lock of the piston. Together, the reservoir and the spindle device arranged on the piston form a particularly compact component. Such an embodiment enables a very flat and at the same time very short structure of the reservoir. This is particularly advantageous for reservoirs for patch pumps. Such systems are applied directly to the user's skin. Small dimensions are particularly advantageous for such applications. Handling is also made easier for the user, because now the reservoir and the spindle device are connected to each other and form a replaceable unit together. This variation has a simple design and includes only a small number of components, is easy to install and inexpensive to make.

According to a preferred embodiment, it is particularly advantageous if the length ratio L/D—wherein L is a longitudinal axis of the oval cross section of the piston and D is a greatest distance between sealing points on the piston—is greater than 0.5 and less than 2.5. For insulin pumps, standard ampoules have volumes from 1.5 cc to 3.5 cc. In conventional metering apparatuses, the sealing points an the piston tend to be very close to each other, so that it is possible to receive the most compact ampoule possible with a minimum lengthwise dimension. Accordingly, length ratio L/D is very large, greater than 3.0 for standard commercial reservoirs. However, it has been found that for the special case of pistons with oval cross sections a ratio of less than 2.5 is advantageous, because such a length ratio L/D prevents the plug from rocking when medication fluid is discharged. Rocking of the piston can have a negative effect on the accuracy with which the medication is dispensed. Moreover, for length ratios L/D of less than 2.5 it is possible to arranged the spindle device directly on the piston. According to claim 4, a reservoir can be manufactured that is compact and yet has a spindle device integrated in the piston. Additionally, it is possible to prevent the plug from rocking with a favourable length ratio of less than 2.5. Reservoirs with length ratios of less than 0.5 have good properties with regard to rocking of the piston, but they also have an unfavourable lengthwise dimension. Reservoirs with a length ratio less than 0.5 are not suitable for compact metering apparatuses. The second displacement stage may preferably be equipped with a blocking element in the lower portion thereof, which prevents the second displacement stage from rotating by means of the reservoir wall. Thus, the reservoir wall also functions as an element of the rotational lock for the second displacement stage. Such a configuration further simplifies the construction of the spindle device, and handling is also made easier for the user. According to this variant, the second displacement stage no longer has to be brought into engagement with a fixed housing to prevent the displacement stages from rotating. According to such a preferred variant, the second displacement stage is prevented from rotating by the wall of the reservoir, for this purpose according to a further preferred embodiment a flank serves as a blocking element. The user only has to create the coupling between the entrainment rod of the drive device and the drive stage of the spindle device, and then connect the reservoir in non-rotating manner to a housing. According to such a preferred embodiment, an axial limit stop is needed for the second displacement stage, and according to a further preferred embodiment this limit stop is advantageously conformed on the entrainment rod. In handling step, a partly filled reservoir is brought into engagement with the entrainment rod and the reservoir is fixed to a solid housing by the user, bayonet connections being well suited for this purpose, for example. Now when the drive stage is driven, the second displacement stage moves backwards towards the axial limit stop thereof arranged on the entrainment rod. A force sensor arranged on the drive device for the decrease in an axial force may thus detect the point when the spindle device reaches the limit stop. When this point has been reached, the spindle device and the reservoir are ready for priming of a fluid path or discharge of a medication fluid. The arrangement defined in this preferred embodiment serves to further simplify handling for the user, as the metering apparatus may cooperate with an electronic controller by which it may be brought into a starting state for priming or discharge automatically.

The user is thus relieved of the laborious task of aligning the drive spindle and the piston in order to connect the piston and the drive spindle manually, as is known for conventional insulin pumps. For example, the Spirit Plus insulin pump produced by Roche Diabetes Care GmbH is equipped with a permanent spindle device having a plug plate for connection with a reservoir piston. With partly filled reservoirs, in a first handling step the user must extend the spindle device using a visual estimate as a guide, so that in a second handling step he can connect the piston and the spindle with each other via a threaded union. Connecting the piston and the plug plate is time-consuming and laborious for the user, and if the piston and the plug plate are not aligned correctly air may be aspirated or medication fluid may be discharged. According to a further preferred embodiment, it is also advantageous if the reservoir wall is constructed as the housing and may be connected directly to the first housing. In this way, a lean, compact metering apparatus may be created. This variation is particularly advantageous for patch pumps, which must be worn directly on the skin and therefore must not spread over the user's body too much. A pump with such a design would be perceived by the user as little more than a minor irritation.

According to a preferred embodiment, it is advantageous if the mounting for the reservoir on the housing and the axial limit stop for the second displacement stage are located at the same level or almost exactly the same level along a common longitudinal axis. The housing and the spindle device should also have a similar coefficient of linear thermal expansion. This advantageous variant makes it possible to compensate for temperature fluctuations and thus minimise over- and under-delivery due to thermal variations. In conventional pumps the spindle devices are made predominantly from metal. On the other hand, the reservoir wall and the pump housing are made from plastic. When temperatures vary, differences arise between the coefficients of linear thermal expansion of the plastic components and the metal components. The different coefficients of linear thermal expansion of the components can lead to over- or under-delivery. This preferred variant makes it possible to minimise the over- or under-delivery by ensuring that the components expand equally, the spindle device from its limit stop to the piston and the wall of the reservoir from its fixing point to the piston. It is therefore advantageous if the fixing point of the reservoirs and the limit stop for the spindle device conformed on the entrainment rod are located as closely a possible to the same level along the longitudinal axes thereof.

According to a preferred embodiment it is advantageous if the piston or one of the elements of the spindle device may be connected to a drawing rod. This is advantageous for the user, because he is then able to connect the position to the drawing rod as with conventional ampoules, connect the piston to the drawing rod, fill the reservoir and then connect the reservoir to the drive device. The reservoir may be filled in the same way the user knows and is familiar with from known systems. Thus, the user does not have to complete any new handling steps when filling the reservoir.

The spindle device and the reservoir may preferably form a single, replaceable part. This simplifies handling, because it is no longer necessary to connect the spindle device to the reservoir piston. Since the spindle device can only be brought into a starting state with the aid of special tools, the user can only use a reservoir once. This serves to improve the service life of the metering apparatus, because a new spindle device is also used with each new reservoir. Accordingly, the spindle device according to this preferred embodiment is not exposed to any wear. It is known that spindle devices where are attached permanently to the pump in conventional insulin pumps are subject to considerable wear. Wear of any kind shortens the service life of such metering apparatuses significantly. Since the spindle device defined according to this embodiment can only be used once, safety for the user is increased. If reservoirs are used repeatedly, infections may occur if they become contaminated with bacteria. The reservoir may also develop leaks if it is used multiple times. Leaks result in delivery of insufficient medication fluid to the user. A particularly advantageous embodiment is also disclosed.

According to a preferred embodiment, it is advantageous if the spindle device alone is designed as a replaceable part. For example, the spindle device defined in claim 3 may be designed as a separate part with different displacement paths. In this way, it is possible to create a modular construction approach in which the same drive device may be combined with different spindle devices and different reservoirs. This in turn helps to result the design engineering and development effort for new devices drastically. The spindle device may also preferably be coupled permanently to a drive device. Devices that are used only once for administering medication fluid are known. For devices of such kind, it is expedient to couple the drive device permanently to the spindle device. At this point, it should be noted that embodiments are conceivable in which the drive device and the spindle device are coupled to one another permanently and the spindle device may be used multiple times.

### BRIEF DESCRIPTION OF THE DRAWING

In the following, exemplary embodiments of the invention will be described greater detail with reference to the drawing, wherein:

FIG. 1 shows a longitudinal cross section of a first embodiment of a spindle device according to the invention connected to a prefilled glass ampoule before it is coupled to a drive device,

FIG. 2a shows a longitudinal cross section of the system of FIG. 1 in the coupled state in a starting position,

FIG. 2b shows a longitudinal cross section of the system of FIG. 2a in an extended state,

FIG. 3 shows a longitudinal cross section of a second embodiment according to the invention with a spindle device for an oval reservoir integrated with a piston in a starting state before it is coupled to a drive device,

FIG. 4a shows a longitudinal cross section of the system of FIG. 3 in a starting position in the coupled state,

FIG. 4b shows a longitudinal cross section of the system of FIG. 4a in an extended position,

FIG. 5a shows a longitudinal cross section of the system of FIG. 3 in a starting position in the coupled state with a partly filled reservoir,

FIG. 5b shows a longitudinal cross section of the system of FIG. 5a in the coupled state after the second displacement stage has travelled as far as a limit stop,

FIG. 6a shows a cross section transverse to the longitudinal axis for the system of FIG. 3 of a first embodiment of a blocking element for the second displacement stage in a starting position,

FIG. 6b shows a cross section transverse to the longitudinal axis of the blocking element of FIG. 6a supported on a wall of the reservoir,

FIG. 6c shows a cross section transverse to the longitudinal axis for the system of FIG. 3 of a second embodiment of a blocking element for the second displacement stage in a starting position,

FIG. 6d shows a cross section transverse to the longitudinal axis of the blocking element of FIG.

6c supported on a wall of the reservoir,

FIG. 7 shows a cross section transverse to the longitudinal axis of the reservoir of FIG. 3 coupled with the drive device,

FIG. 8 shows a longitudinal cross section of the reservoir of FIG. 3, wherein the spindle device is connected to a drawing rod,

FIG. 9a shows the replaceable spindle device of FIG. 1 after it has been used,

FIG. 9b shows a longitudinal cross section of the replaceable spindle device of FIG. 9a after it has been used, and

FIG. 9c shows a longitudinal cross section of the reservoir of FIG. 3 with integrated spindle device after it has been used.

### DETAILED DESCRIPTION

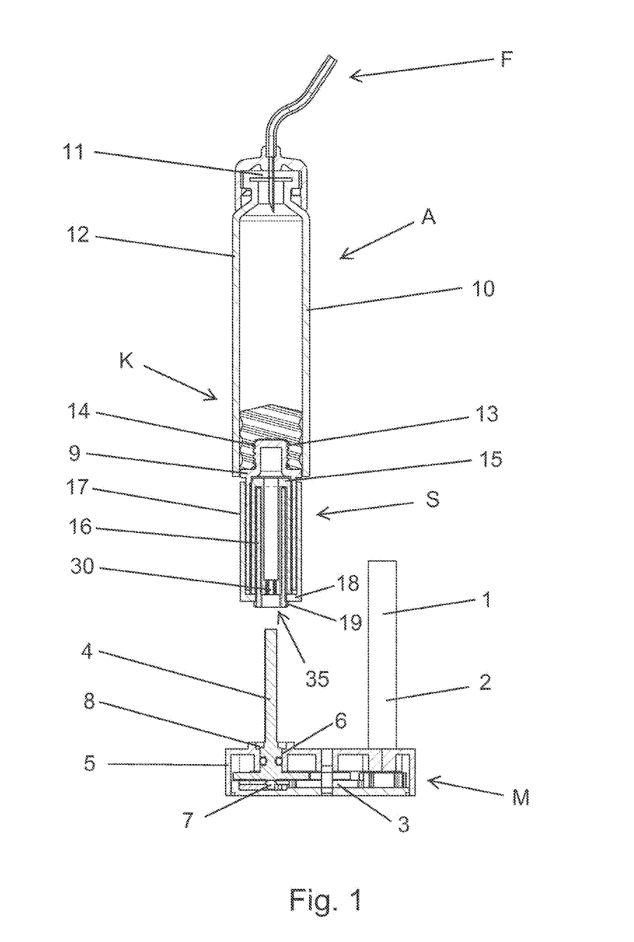

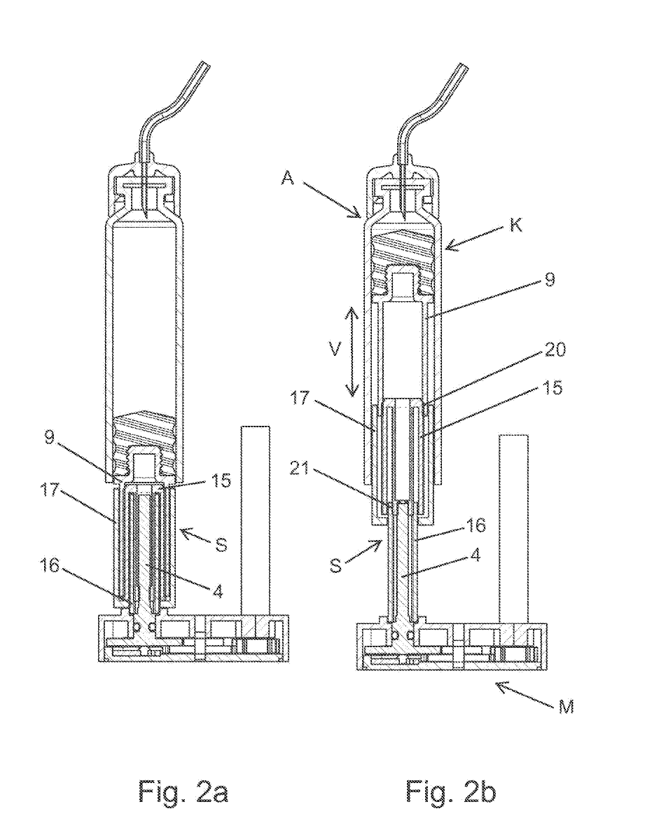

FIG. 1 represents a first embodiment of a spindle device S according to the invention. FIG. 1 shows an arrangement such as is found in an insulin pump, for example. In such a pump, a metering apparatus comprises a drive device M, a replaceable spindle device S and a reservoir A containing medication fluid. Only the start of a fluid path F which connects the pump to the user is shown here. In conventional insulin pumps, spindle device S is connected directly to drive device M and cannot be replaced. Examples of this type are the D-TRONplus and Accu-Chek pumps manufactured by Roche Diabetes Care GmbH. The drive device M shown in FIG. 1 includes a motor 1, a planetary gear system 2, a reducing spur gear 3 that drives an entrainment rod 4, functioning as a coupling member in rotary manner, and a housing 5. Entrainment rod 4 functions as an interface element between drive device M and spindle device S. Entrainment rod 4 has a circular cross section at its sealing point and is therefore able to be insulated from housing 5 by means of an o-ring. The narrow cross section of entrainment rod 4 allows insulation to be assured against inner housing wall 6 efficiently and with low friction losses. In this way, foreign fluid is effectively prevented from getting into the gear system space. Entrainment rod 4 is supported axially against a housing rear wall via a force sensor 7 arranged therebetween. The purpose of force sensor 7 is a measure an axial force acting on entrainment rod 4. An axial limit stop 8 conformed on entrainment rod 4 serves as the limit stop for spindle device S. Thus, an axial force acting on spindle device S is transmitted entrainment rod 4 to force sensor 7. The measurement of the spindle force is used to detect when spindle device S reaches its limit stop, to detect a blockage in the fluid path and to monitor further states of the pump. The spindle device S according to the invention shown in FIG. 1 has a first displacement stage 9, which is connected to a piston K of a reservoir A. In the embodiment shown, reservoir A comprises a prefilled glass ampoule 10 with a septum 11 connection to a fluid path F, a round glass body 12 and a rubber piston K, said piston being furnished with an internal thread 13 so that glass ampoule 10 and spindle device S may be joined to each other. For a threaded connection with piston K, the first displacement stage 9 of spindle device S has an external thread 14 which engage in the internal thread 13 of piston K. In a first handling step, the user connects spindle device S to piston K by screwing the two elements together. In a second handling step, the system consisting of glass ampoule 10 and spindle device S is inserted axially in an ampoule compartment for seating in a housing. This produces a coupling operation between drive device M and spindle device S, in which entrainment rod 4 is coupled with a drive stage 15 of spindle device S. At the same time, a second displacement stage 16 of spindle device S is connected in non-rotating manner to the fixed housing. After drive device M has been coupled with spindle device S, the pump is ready to be connected to a fluid path F. Only the start of fluid path F is shown in FIG. 1. The purpose of this path is to assure the fluid connection between ampoule A and the user. In general, fluid path F may include an adapter that may be attached to ampoule A for connecting ampoule A to fluid path F, a catheter tube and a port with a cannula at the end of the catheter tube for connecting to the user. When motor 1 turns, entrainment rod 4 performs a reduced rotating motion, which in turn drives drive stage 15 in a rotary manner. To ensure that displacement stages 9 and 16 are only moved axially, first displacement stage 9 and second displacement stage 16 are connected to each other in non-rotating manner via a concentrically arranged cylindrical sleeve 17. Sleeve 17 is equipped with radial dogs 18, which engage in corresponding longitudinal grooves 19 on the displacement stages. In the arrangement according to FIG. 1, the first displacement stage 9, second displacement stage 16 and sleeve 17 are connected to each other in non-rotating manner. A first spindle drive 20 consisting of threads is positioned between first displacement stage 9 and drive stage 15. A second spindle drive 21 consisting of threads is also positioned between second displacement stage 16 and drive stage 15. The two spindle drives 20 and 21 are designed to rotate in opposite directions, so that the one spindle drive is made up of right-handed threads and the other is made up of left-handed threads. In a first step, an axial longitudinal play between spindle device S and drive device M must be travelled, during which process the second displacement stage 16 is displaced backwards towards its assigned limit stop 8 arranged on entrainment rod 4. When second displacement stage 16 reaches its axial limit stop 8, the longitudinal play between spindle device S and drive device M has been cancelled. The pump is now in a starting state for dispensing medication. This starting state is shown in FIG. 2a. If drive stage 15 is driven to rotate further, first displacement stage 9 moves away from drive stage 15 and drive stage 15 moves away from the second displacement stage 16, which is positioned at its limit stop 8. Since both spindle drives 20 and 21 are driven at the same time, any thread play in the two spindle drives is compensated for at the same time. Therefore, means for compensating for thread play are not required. The threads may have a pitch of 0.4 mm per revolution, for example. Thus, when drive stage 15 makes one revolution, spindle device S and therewith also piston K are displaced by 0.8 mm per revolution. The spindle device of the D-TRONplus pump has a pitch of 1.2 mm per revolution, for example, and its spindle drives rotate in the same direction. The variant of spindle device S according to the invention shown in FIG. 1 is compact, and its radial extent is so small that it is particularly well suited for dispensing prefilled glass ampoules containing insulin. The ampoules generally have a narrow ampoule body, of which the internal diameters may be as little as just 8 to 10 mm. If drive stage 15 is turned further still, spindle device S bears on piston K in the direction of advance and ejects medication fluid. Said fluid is then administered to the user. For the administration, any delivery profiles are conceivable. In insulin pump therapy, insulin is delivered in two different ways, basal and bolus delivery. When piston K reaches a top position, ampoule A is emptied. This fully extended state of spindle device S is shown in FIG. 2b. In this state, the engagement between entrainment rod 4 and drive stage 15 is minimal. The user can easily remove ampoule A and the spindle device S connected to it via piston K from the ampoule compartment. Then, the pump can be loaded with a new, prefilled ampoule A and a new spindle device S. Since first displacement stage 9, sleeve 17 and second displacement stage 16 are connected to each other in non-rotating manner and sleeve 17 surrounds drive stage 15, the user is not able to bring a spindle device S into the starting state again once it has been used. Consequently, he is obliged to use a new spindle device S. The use of a new spindle device S for each new ampoule A increases patient safety and extends the service life of drive device M. The drive devices that are coupled with permanent spindle devices in conventional systems, such as those used in the D-TRONplus and Accu-Chek pumps for example, must complete several hundred strokes with absolute reliability in the course of their service lives. The spindle devices of conventional systems which are connected permanently to the pump are subject to very strong wearing forces and often fail before the end of their guaranteed service life.

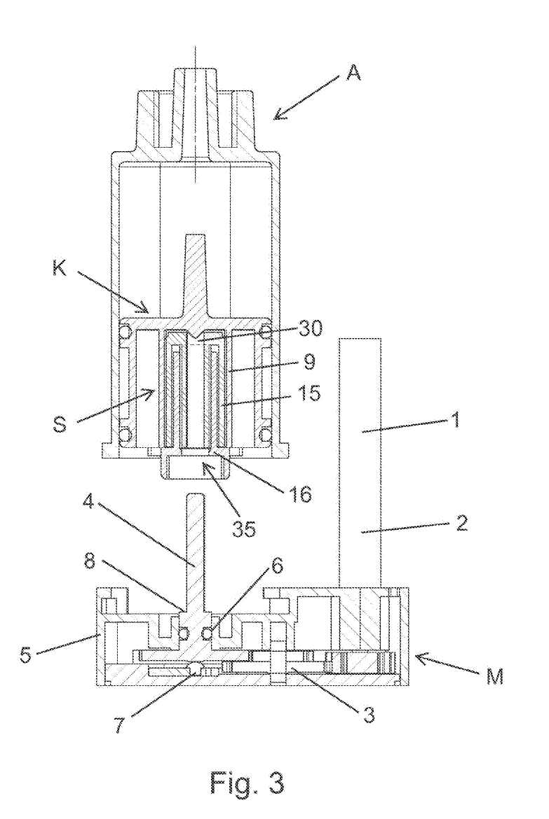

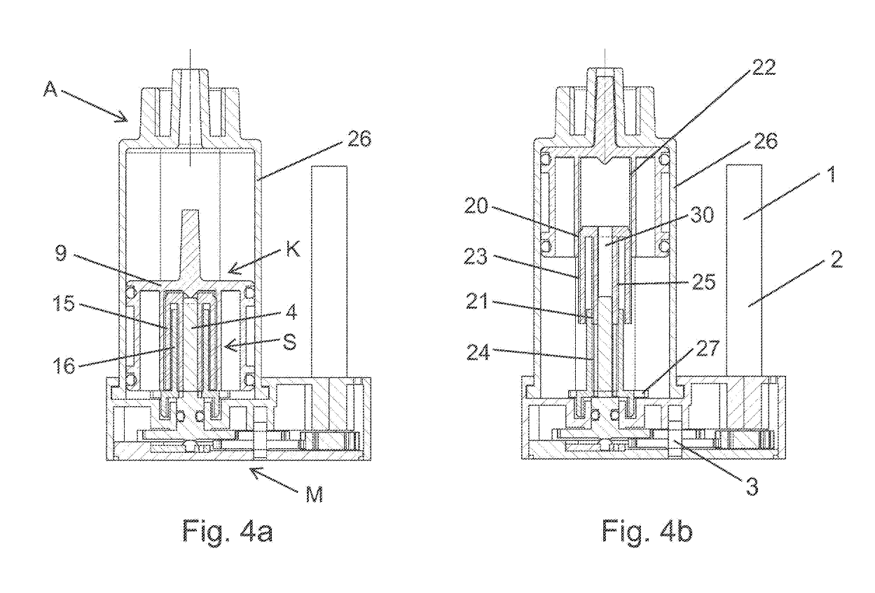

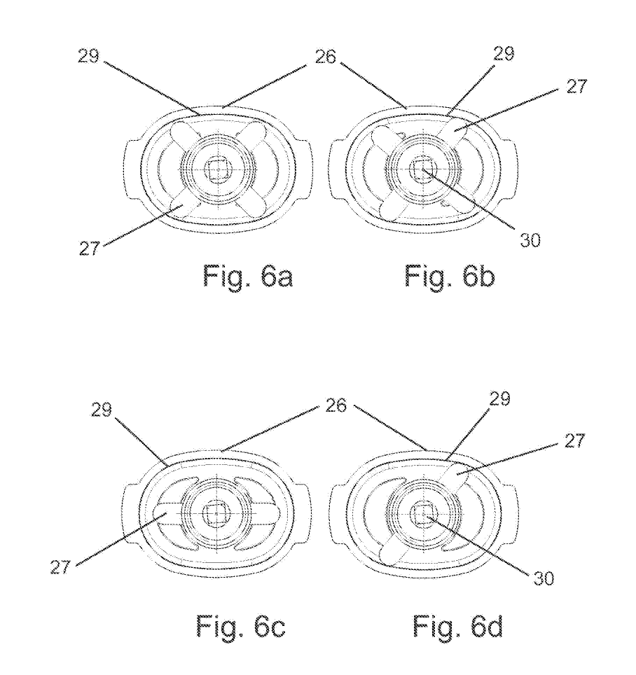

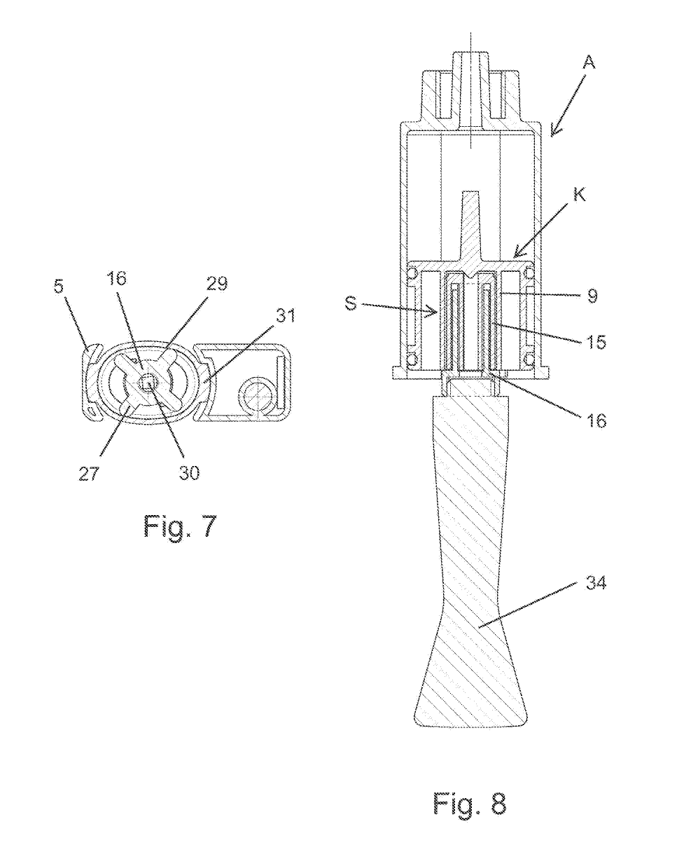

FIGS. 3 to 8 illustrate a second embodiment according to the invention. This offers particularly favourable handling for the user, because spindle device S is integrated directly in a piston K of a reservoir A. FIG. 3 shows the spindle device S arranged on piston K and the oval reservoir A before it is coupled to a drive device M. Piston K serves as first displacement stage 9 of spindle device S and in addition has an oval cross section. A reservoir A configured in this way is advantageous for applications in which a flat pump contour is required. An internal thread 22 on first displacement stage 9 cooperates with an external thread 23 conformed on a drive stage 15 to create a first spindle drive 20. Drive stage 15 in turn is connected to a second displacement stage 16 via a further spindle drive 21 that is formed from threads 24 and 25. The two spindle drives 20 and 21 rotate in opposite directions for this embodiment as well. Oval reservoir A serves as an element for the rotational lock 26 of piston K. A spindle drive moment acting on first displacement stage 9 and thus also on piston K is absorbed by reservoir A. The bottom end of second displacement stage 16 is furnished with a radial blocking element 27 in the form of a flank 28. Embodiments of blocking element 27 in a starting position and a blocking position are illustrated in FIGS. 6a, 6b, 6c and 6d. The blocking element 27 of FIGS. 6a and 6c has a radial extension that is larger than the smallest distance between opposing walls of reservoir A. A spindle drive moment acting on second displacement stage 16 is thus transmitted to reservoir A via blocking element 27, as is shown in FIGS. 6b and 6d. Second displacement stage 16 is thus prevented from rotating, wherein a wall 29 of reservoir A functions as a reaction member for the drive moments produced in spindle drives 20 and 21 for both displacement stages 9 and 16. Wall 29 of reservoir A thus corresponds to the element for the rotational lock 26. The user is easily able to couple reservoir A including the integrated spindle device S with drive device M. In doing so, he inserts a profiled entrainment rod 4 arranged on drive device M into an axial hole 30 formed in drive stage 15. Square or stellate cross sections, for example, are suitable for the profiling of entrainment rod 4 and hole 30. On the other hand, reservoir A is connected in non-rotating manner to a housing 5 of drive device M via a bayonet connection 31. In this case, wall 29 of reservoir A is itself constructed as the external housing, so that the pump has a very flat contour. When entrainment rod 4 rotates, driven by motor 1, and so propels drive stage 15, as first displacement stage 9 piston K moves away from drive stage 15. At the same time, drive stage 15 itself moves away from the second displacement stage 16, which is positioned at the axial limit stop 8 thereof. The forward motion generated thereby is equivalent to a thread pitch per revolution for drive stage 15 and two thread pitches per revolution for piston K. Piston K is thus displaced by one thread pitch per revolution relative to drive stage 15. In FIG. 4a, the system is in a starting state. According to FIG. 4a, second displacement stage 16 is positioned at its limit stop 8 conformed on entrainment rod 4. In corresponding manner, in FIG. 4b piston K is in a top position and spindle device S is in a retracted position. Drive device M will not be described again here, since its construction is the same as that of the first embodiment. Particularly this property, which enables different spindle devices 5 to be combined easily with a single drive device M, thereby producing a modular construction, represents a significant advantage.

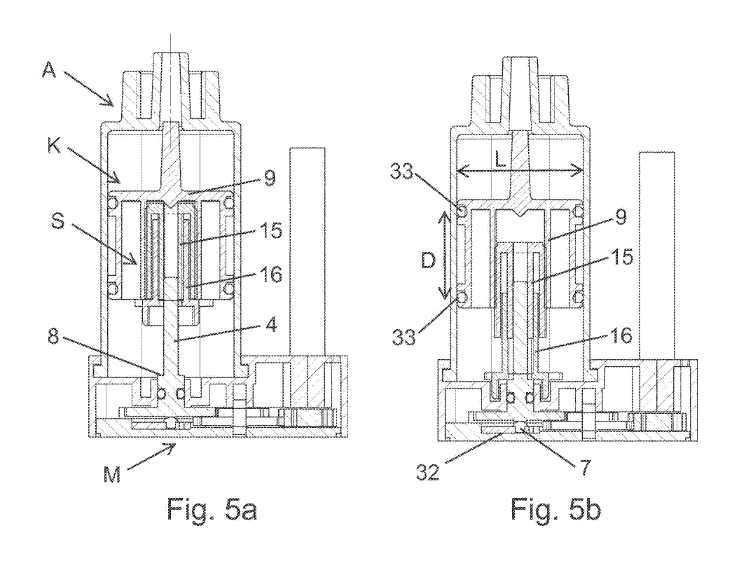

The reservoir A represented in FIGS. 3 to 8 may also be only partly filled, wherein the filling constitute 50 to 100% of the maximum volume. A reservoir A that is 50% full is shown in FIG. 5a. Here, entrainment rod 4 has a minimal overlap with drive stage 15. An axial lengthwise separation exists between the limit stop 8 arranged on entrainment rod 4 for the second displacement stage 16 and the second displacement stage 16. In the starting position shown in FIG. 5a, a rotating motion of drive stage 15 causes second displacement stage 16 to be displaced backwards. In this operation, piston K is not displaced due to the static friction acting between piston K and reservoir A. Now when second displacement stage 16 reaches its limit stop 8, a force sensor 7 for measuring an axial force arising on entrainment rod 4 can determine that spindle device S has reached the limit stop and stop drive device M. For this purpose, force sensor 7 is arranged on drive device M, wherein entrainment rod 4 is supported on the housing via the force sensor 7 arranged between entrainment rod 4 and a housing rear wall 32. After second displacement stage 16 has reached its axial limit stop 8, the system is in the position shown in FIG. 5b. In a further step, the user primes the fluid path F. In this operation, the user ensures that drive device M and reservoir A are connected to each other correctly, and that spindle device S is supported on its limit stop. If these required conditions are satisfied, priming causes fluid path F to be filled. For safety reasons, the user is not connected to the pump during priming. The user continues priming until the first discharge of medication is observed in the form of a drop. At this point, the user stops priming the fluid path and connects the pump to his body via a port. The port has a cannula for introducing the medication fluid into the body.

The embodiment according to the invention shown in FIGS. 3 to 8 is extremely advantageous with regard to handling, size and safety. The advantages that can be realised with it will be listed and discussed again here.

The reservoir A connected to spindle device S represented in FIGS. 3 to 8 is particularly well suited for use in a patch pump in insulin pump therapy. Reservoir A of FIGS. 3 to 8 has a fluid volume of 2 cc. Its external diameter is just 11 to 14 mm. The fact that spindle device S is integrated in piston K means that the axial lengthwise dimension of reservoir A is very advantageous. It is thus possible to reduce the axial length of the patch pump shown in FIGS. 3 to 8 considerably, and it may be 45 to 50 mm for the embodiment. This length dimension must be compared with those of conventional insulin pumps, which have lengths from 70 to 120 mm and depths from 18 to 22 mm. In contrast, commercially available patch pumps have lengths from 52 to 62 mm and depths from 14 to 18 mm. The reservoir A shown in FIGS. 3 to 8 further has an oval cross section of 125 m̂2, a stroke of 16 mm and a travel path V of 8 mm per stage. The thread pitch per stage is 0.4 mm per revolution.

The embodiment shown in FIG. 3 also has a length ratio L/D of 1.32, wherein L is the longitudinal axis of the piston cross section, and in this embodiment has a value of 13.9 mm. D is the greatest distance between sealing points 33 of piston K and in this embodiment has a value of 10.5 mm. With a length ratio L/D of this order, it is possible to create a particularly compact piston K with an integrated spindle device S. Piston K also has a good guide formed by seals within reservoir A. In this context, the seals may be in the form of o-rings 33, and may have two or more sealing points. It is also conceivable to attach the sealing points to piston K directly in an injection moulding process. Particularly for an oval piston, the L/D ratio is critical for good guidance and precise dosing. For particularly favourable length ratios L/D, piston K does not rock inside reservoir A. The piston will rock of the greatest distance between sealing points 33 is not large enough and the L/D ratio consequently exceeds a limit value. For the embodiment shown in FIG. 3, the limit value is 2.5. If the L/D ratio is less than 0.5, the greatest distance between sealing points 33 becomes so great that reservoir A becomes disadvantageously long. For a reservoir with an integrated spindle device S and a volume of 2 to 3.5 ml, it is particularly favourable if length ratio L/D is in a range from 0.5 to 2.5. In the preferred range, reservoir A with an integrated spindle device S has a short structure, and piston K is securely guided inside reservoir A. Rocking during displacement is prevented and delivery of medication fluid can be dispensed with great accuracy.

A further advantage of the device is the simplicity of its design, because the spindle device is made from just three parts. A first component can be dispensed with of the embodiment of FIGS. 3 to 8 because the first displacement stage 9 is conformed directly on piston K. The oval reservoir A also assumes the function of the rotational lock for first displacement stage 9 and second displacement stage 16. This makes it possible to dispense with a second component of the construction. In the embodiment of FIGS. 3 to 8 wall 29 of reservoir A serves as an element for rotational lock 26. Consequently, the embodiment shown in FIGS. 3 to 8 comprises only four components for reservoir A and spindle device S. These are the wall 29, piston K, drive stage 15 and second displacement stage 16. The embodiment shown in FIGS. 3 to 8 easy to produce and install, and can be manufactured inexpensively. Installation is effect simply be screwing the two threaded joints on piston K together and then piston with its preinstalled spindle device S is inserted in reservoir A.

Since spindle device S cannot be brought into a starting state manually by a user, the reservoir A with integrated spindle device S shown in FIGS. 3 to 8 can only be used once. In insulin pump therapy, it is recommended to use a reservoir only once. However, conventional reservoirs share a common drawback in that they can be used more than once. When they are used more than once, the user may be exposed to contaminants and in the worst case infections. Problems with leaking from the reservoir may also occur, which in result in smaller delivery volumes of the medication. Furthermore, the replacement of reservoir A and thus also of spindle device S prolongs the service life of drive device M. Drive device M must generate the spindle driving torque equivalent to the driving torques of both spindles necessary for displacing the medication fluid. Since a new spindle device S integrated on piston K is also used for each reservoir A used, the spindle driving torque remains constant for the service live of the metering apparatus. In addition, driving device M is easy to seal effectively, as shown in FIGS. 3 to 8, and this also helps to increase the service life of the metering apparatus.

The spindle device S shown in FIGS. 3 to 8 also does not require any means for compensating for play, since both spindle drives 20 and 21 are driven at the same time, wherein the one spindle drive consists of left-handed threads and the other consists of right-handed threads. With this arrangement, both thread plays can be compensated simultaneously by rotating drive stage 15. Means for play compensation are thus not needed in the embodiments of FIGS. 3 to 8, and this also results in a further reduction of the axial length of spindle device S. For the embodiments shown in FIGS. 3 to 8, both drive stage 15 and first displacement stage 9 a displaced with advancing movements simultaneously with medication is delivered. This synchronised displacement of both stages means that there is no need for limit stops between the stages to prevent the spindle stages from becoming separated. By comparison, such limit stops are indispensable in the prior art. The spindle device of the D-TRONplus pump includes such limit stops because its stages cannot be extended at the same time, they must be extended consecutively. The limit stops prevent the stages from travelling to their full extension, which would result in the stages separating completely. Since there is no need for either means for play compensation or limit stops for displacement stages 9 and 16 and drive stage 15 the axial length can be reduced substantially.

Further advantages for the embodiment represented in FIGS. 3 to 8 will be outlined briefly here. Spindle device S does not need to be moved backwards when reservoir A is replaced, which makes handling easier for the user. Moving the spindle device of the D-TRONplus pump back as a reference takes several minutes. Reservoir A is filled in the same way the user knows from the existing state of the art. This is beneficial for the user because he does not have to learn any new handling steps. The user uses a drawing rod 34 to fill reservoir A. This may e coupled directly or indirectly to piston K. In FIG. 8, reservoir A of FIG. 3 is connected to a drawing rod 34. In this context, drawing rod 34 has an external thread, which the user connects to an internal thread conformed on second displacement stage 16. Drawing rod 34 and piston K of reservoir A are now connected to each other and the user can proceed to fill reservoir A. The steps for filling reservoir A are listed here and are: connect drawing rod 34 to piston K, make fluid connection between a storage vessel with medication and reservoir A by means of a connecting adapter, move piston K to an upper position and force the air out of reservoir A into the storage vessel, then move piston K back to its starting position, causing the medication fluid to flow form the storage vessel into reservoir A, filling reservoir A. The reservoir A shown in FIGS. 3 to 8 can be connected to fluid path F in various ways. For example, reservoir A may have a septum and it may be connected to a needle of an adapter or it may itself have a needle for connection arranged inside its housing. It is also conceivable that a Luer lock is provided to connect a catheter tube directly to reservoir A.

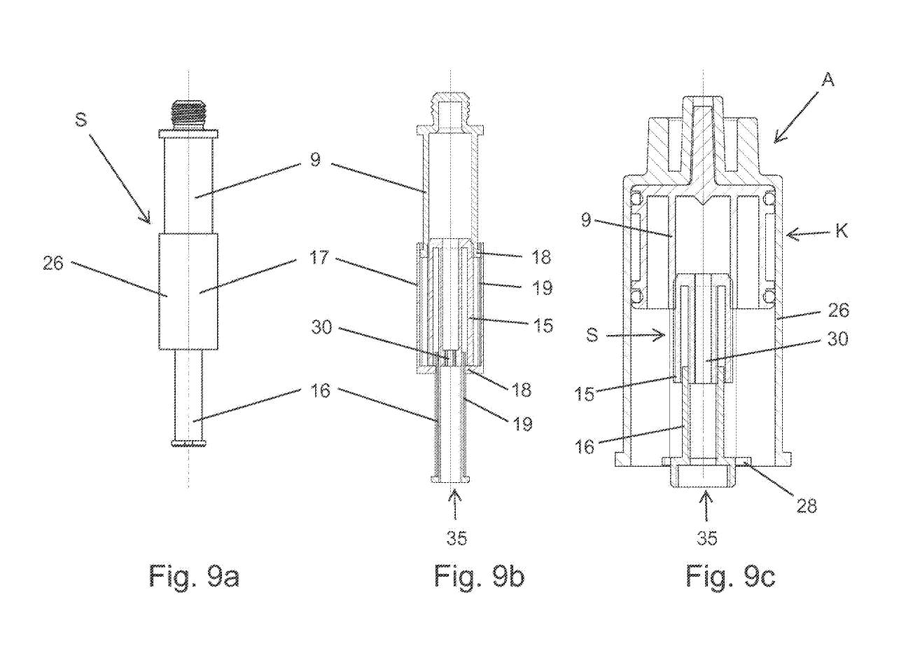

In FIGS. 9a, 9b and 9c, spindle devices S of the embodiments of FIGS. 1 to 8 are shown after they have been used. After use, the spindle devices are fully extended. In FIGS. 9a and 9b, the spindle device S of the embodiment of FIGS. 1 and 2 is shown. This spindle device S may be separated both from drive device M and from reservoir A. FIG. 9c illustrates the spindle device S that is connected in fixed manner to reservoir A as shown in FIGS. 3 to 8. Reservoir A and spindle device S of FIG. 9c can only be separated from drive device M. It is quite evident that the user cannot use the embodiments shown in FIGS. 9a, 9b and 9c a second time. Drive stage 15 is permanently surrounded radially by rotational locking element 26 and is only accessible to the user from the side 35 opposite piston K with the aid of tools. Displacement stages 9 and 16 are connected in non-rotating manner with rotational locking element 26. For the spindle devices S shown after use in FIGS. 9a, 9b and 9c, it is not possible for the user to return the spindle devices S to a starting state and use them again. This improves safety for the user and prolongs the service life of the metering apparatus, since a new spindle device S is also used whenever a new reservoir A is used.

It is evident from FIGS. 9a, 9b and 9c that the two displacement stages 9 and 16 are connected to each other in non-rotating manner via the surrounding rotational lock element 26. For the embodiment of FIGS. 9a and 9b, the non-rotating connection is formed by dogs 18 and longitudinal grooves 19, for the embodiment of FIG. 9c, the rotational lock of displacement stages 9 and 16 is assured by the oval shape of the wall 29 of receptacle A. It is therefore sufficient that for spindle device S one of the three elements 9, 16 or 26 is connected to the housing in non-rotating manner. For the embodiment of FIGS. 9a and 9b, this is realised via second Displacement stage 16, for the embodiment of FIG. 9c via element 26.

For both embodiments shown in FIGS. 1 to 9, drive stage 15 is formed by two cylindrical sleeves. In this context, the cylindrical sleeves are connected to each other in non-rotating manner by their upper frontal faces closest to piston K. Drive sleeve 15 is also furnished with the two external threads 23 and 25 for spindle drives 20 and 21. The internal sleeve has hole 30 for coupling with drive device M. The embodiments of FIGS. 1 to 9 represent parallel embodiments of the spindle device S. In parallel embodiments according to FIGS. 1 to 9, two spindle stages of spindle device S are extended simultaneously, whereas in serial embodiments only one spindle stage is displaceable. For the examples of FIGS. 1 to 9, drive stage 15 and one of the two displacement stages 9 or 16 are displaced simultaneously. After the metering apparatus has been charged with a completely or partly filled reservoir A, the first action must be to compensate for axial longitudinal play between the spindle device S and the drive device M. In order to compensate for the axial longitudinal play between spindle device S and the limit stop of drive device M, spindle device S first moves backwards. In this context, both drive stage 15 and second displacement stage 16 move backwards to towards limit stop 8. After the compensation for longitudinal play, the metering apparatus is ready for priming the fluid path F. During priming and the subsequent medication delivery, drive stage 15 and the first displacement stage 9 travel simultaneously in the direction of advance. Second displacement stage 16 is braced against its fixed limit stop 8 and then functions as a reaction stage. In this context, it absorbs the turning moment produced in spindle drive 21 and transmits the axial force exerted by spindle device S to limit stop 8. Since spindle device S is displaceable both backwards to compensate for longitudinal play and in the direction of advance for delivering the medication, handling is made significantly simpler for the user. The pump controller is able to take charge of and carry out longitudinal play compensation automatically.

It should be noted at this point that further embodiments of the invention are conceivable, and the embodiments illustrated here are not exhaustive. For example, drive stage 15 has an external thread for creating a screwed joint with second displacement stage 16. However, it is entirely conceivable to provide drive stage 15 with an internal thread and second displacement stage 16 with an external thread for second spindle drive 21. Modifications of such kind do not result in new and inventive solutions. In the same way, a reversal of the spindle device S does not constitute a new or inventive solution, in which the second displacement stage is directed towards the piston and the first displacement stage is directed towards the drive device M. Since the three elements—first drive stage 9, second drive stage 16 and the rotational lock element 15—are connected to each other in non-rotating manner, it is sufficient to connect one of the three elements, 9, 16 or 15 with a fixed housing to ensure that none of the elements 9, 16 and 15 can rotate. In FIGS. 1 to 9, only the essential features of the metering apparatus are shown. These are the drive device M, the spindle device S and the reservoir A. Other components and parts such as batteries, displays, housings, fluid paths and the like which are not significant for the purposes of the invention were not represented. The embodiments shown in FIGS. 1 to 9 are advantageous for patch pumps, conventional medication administration systems such as insulin pumps and pens. The invention is particularly advantageous for compact devices for delivering medication fluid. Telescopic spindle devices are always more compact than single-stage spindle devices and consequently have a favourable lengthwise dimension. Further applications and devices for which precise delivery of medication fluid is important are also conceivable.

### LIST OF REFERENCE SIGNS

S Spindle device

M Drive device

A Reservoir

K Piston

F Fluid path

1 Motor

2 Planetary gear system

3 Reducing spur gear

4 Entrainment rod

5 Housing

6 Inner housing wall

7 Force sensor

8 Limit stop

9 First displacement stage

10 Glass ampoule

11 Septum

12 Glass body

13 Internal thread

14 External thread

15 Drive stage

16 Second displacement stage

17 Sleeve

18 Dog

19 Longitudinal groove

20 First spindle drive

21 Second spindle drive

22 Internal thread

23 External thread

24 Thread

25 Thread

26 Rotational lock element

27 Blocking element

28 Flank

29 Wall

30 Hole

31 Bayonet connection

32 Housing rear wall

33 Sealing point

34 Drawing rod

35 Opposing frontal face

1. Spindle device for a piston (K), which is supported in a reservoir(A) containing a medication fluid, wherein the spindle device (S) comprises:
a) a first non-rotatable displacement stage (9) with a thread (22),
b) a second non-rotatable displacement stage (16) with a thread (24),
c) a drive stage (15) arranged between the first and second displacement stages (9,16) having two threads (23,25), wherein the one thread (23) is in engagement with the thread (22) of first displacement stage (9), thus forming a first spindle drive (20), and the second thread (25) is in engagement with the thread (24) of the second displacement stage (16), thus forming a second spindle drive (21), and the threads (22,24) of the first and second displacement stages (9,16) rotate in opposite directions, and wherein the spindle device is designed such that the first displacement stage (9) and the drive stage (15) are displaced simultaneously in the direction of advance,
wherein an element for ensuring a rotational lock (26) surrounding the drive stage (15) axially connects the first and second displacement stages (9,16) to each other in non-rotating manner, and the drive stage (15) of the spindle device (S) can be coupled in non-rotating manner with an entrainment rod (4) of a drive device (M) configured as a coupling member by a frontal face (35) of the spindle device (S) farthest from the piston, wherein the entrainment rod (4) can be introduced into an axial hole (30) formed on the drive stage (15).

2. Spindle device according to claim 1, characterized in that a profiled entrainment rod (4) protruding into the drive stage is constructed as a coupling member for the drive stage (15), wherein in a starting state the entrainment rod (4) and the drive stage (15) have an overlap of at least one displacement path V of a displacement stage (9, 16).

3. Spindle device according to claim 1 or 2, characterized in that a cylindrical sleeve (17) is constructed as a rotational lock element (26), and the second displacement stage (16) can be mounted in non-rotating manner directly or indirectly on a fixed housing (5).

4. Spindle device according to claim 1 or 2, characterized in that the spindle device (S) for the piston (K) is arranged on the piston (K) itself and the piston (K) is constructed as the first displacement stage (9), wherein the piston (K) and the reservoir (A) have oval cross sections and a wall (29) of the reservoir (A) is constructed as a rotational lock element (26).

5. Spindle device according to claim 4, characterized in that the length ratio L/D of a longitudinal axis L of the oval piston cross sections to a greatest distance D between sealing points (33) on the piston (K) has a value greater than 0.5 and less than 2.5.

6. Spindle device according to claim 4 or 5, characterized in that the second displacement stage (16) has a blocking element (27) in the lower area thereof, whose maximum radial extension is grater than the smallest distance between opposing interior surfaces of the wall (29) of the reservoir (A), so that the wall (29) of the reservoir (A) functions as an element for the rotational lock (26) of second displacement stage (16).

7. Spindle device according to claim 6, characterized in that the wall (29) of the reservoir (A) is constructed as a reaction element for a blocking element (27) of the second displacement stage (16) in the form of a flank (28), and the second Displacement stage (16) is displaceable to an axial limit stop (8).

8. Spindle device according to claim 7, characterized in that the axial limit stop (8) for the second displacement stage (16) is conformed on the entrainment rod (4), which is driven in rotary manner.

9. Spindle device according to any one of claims 4 to 8, characterized in that the wall (29) of the reservoir (A) itself is constructed as a housing, and the reservoir (A) can be connected and fixed axially with the housing (5).

10. Spindle device according to claim 9, characterized in that the fixation of the reservoir (A) on the housing (5) and the axial limit stop (8) for the second displacement stage (16) are positioned at the same level or almost exactly at the same level along the common longitudinal axis, and the drive stage (15) and displacement stages (9,16) are made from plastic with a coefficient of linear thermal expansion similar to that of the reservoir (A).

11. Spindle device according to any one of claims 4 to 10, characterized in that the piston (K) or one of the elements of the spindle device (S) can be coupled to a drawing rod (34).

12. Spindle device according to any one of claims 4 to 11, characterized in that the spindle device (S) and the reservoir (A) form a single replaceable part.

13. Spindle device according to any one of claims 1 to 3, characterized in that the spindle device (S) alone is constructed as a replaceable part of a metering apparatus.

14. Spindle device according to any one of claims 1 to 11, characterized in that the spindle device (S) is permanently in a coupled connection with the drive device (M).
